(19) United States
(12) Plant Patent Application Publication
(10) Pub. No.: US 2003/0177552 P1
(43) Pub. Date: Sep. 18, 2003

(54) Hybrid tea rose plant named 'KORrabea'

(75) Inventor: Tim-Hermann Kordes, Offenseth-Sparrieshoop (DE)

Correspondence Address: Russell D. Orkin, 700 Koppers Building, 436 Seventh Avenue, Pittsburgh, PA, 15219-1818, US

(73) Assignee: W. Kordes Sohne Rosenschulen GMBH & Co. KG

(21) Appl. No.: 10/100,342

(22) Filed: Mar. 18, 2002

(51) Int. Cl. A01H 5/00

(52) U.S. Cl. PLT/139

(57) ABSTRACT

This invention relates to a new and distinct variety of hybrid tea rose plant named ‘KORrabea’ which has attractive deep red colored flowers and attractive dark green foliage. This new variety is characterized by having a high production of long stems with a long vase life, vigorous and upright growth, and good tolerance to powdery mildew. The variety successfully propagates from softwood cuttings and is very suitable for year round production of cut flowers in commercial glass houses as a flowering cut rose.

### BACKGROUND OF THE INVENTION

The present invention constitutes a new and distinct variety of hybrid tea rose plant named ‘KORrabea’ which was developed by artificially pollinating an unnamed, unpatented seedling with another unnamed, unpatented seedling. The two parents were crossed in the summer of 1999 and the resulting seed was sown in December 1999 in a controlled greenhouse environment. The objectives of the hybridization was to create a new and distinct rose variety for commercial greenhouse culture with the following characteristics:

1. Uniform, large buds that are well turbinated and flowers with good vase-life.

2. Novel flower color.

3. High cut flower production, with medium to long stems.

4. Vigorous, upright growth.

5. Suitability for production from softwood cuttings.

6. Good tolerance to powdery mildew.

7. Suitability for production of cut flowers under greenhouse growing conditions.

This combination of qualities was not present in previously available commercial cultivars of this type and distinguish ‘KORrabea’ from other varieties known to the inventor.

The seeds from hybridization were planted in a controlled environment and evaluations were conducted on the resulting plants. ‘KORrabea’ was selected in a development program in Klein Offenseth, Sparrieshoop, Germany.

Asexual reproduction of ‘KORrabea’ by cuttings was first done in Germany and later in California. The reproductions by cuttings in controlled greenhouse environments have confirmed that distinguishing features of the new cultivar come true, remain stable and are retained through successive propagations. ‘KORrabea’ is a highly productive hybrid tea rose with a vase life greater than 12 days. Under conventional greenhouse conditions in Germany (64° C. nights, 26° C. days), ‘KORrabea’ will produce 160 stems averaging 80 cm to 90 cm long.

The new rose is distinguished from its seed parent by the following combination of characteristics:

1. ‘KORrabea’ has big double flowers, while the unnamed seedling has medium double flowers.

2. ‘KORrabea’ has dark red colored petals, while the unnamed seedling has dark orange petals.

The new variety may distinguished from its pollen parent by the following combination of characteristics:

1. ‘KORrabea’ has bigger flowers and foliage as compared to the unnamed seedling.

2. ‘KORrabea’ has dark red colored petals, while the unnamed seedling has dark pink petals.

### DESCRIPTION OF THE DRAWINGS

The accompanying color illustrations show as true as possible in color photographs of this type, the typical characteristics of the buds, flowers, leaves, stems of ‘KORrabea’.

In photo sheet #1:

FIG. 1 illustrates a young shoot;

FIG. 2 illustrates a bud before opening of the sepals;

FIG. 3 illustrates a bud at the opening of the sepals;

FIG. 4 illustrates a bud at the opening of the petals;

FIG. 5 illustrates a flower during course of opening;

FIG. 6 illustrates an open flower, plan obverse view;

FIG. 7 illustrates an open flower, plan reverse view;

FIG. 8 illustrates a fully open flower, plan obverse view; and

FIG. 9 illustrates a fully open flower, plan reverse view.

In photo sheet #2:

FIG. 10 illustrates a receptacle showing stamens and pistils;

FIG. 11 illustrates a receptacle showing pistils (stamens removed);

FIG. 12 illustrates flower petals detached, outer surface;

FIG. 13 illustrates flower petals detached, inner surface;

FIG. 14 illustrates a bare stem exhibiting thorns and flower attachment;

FIG. 15 illustrates 3 leaflets, upper side;

FIG. 16 illustrates 3 leaflets, under side;

FIG. 17 illustrates 5 leaflets, upper side; and

FIG. 18 illustrates 5 leaflets, under side.

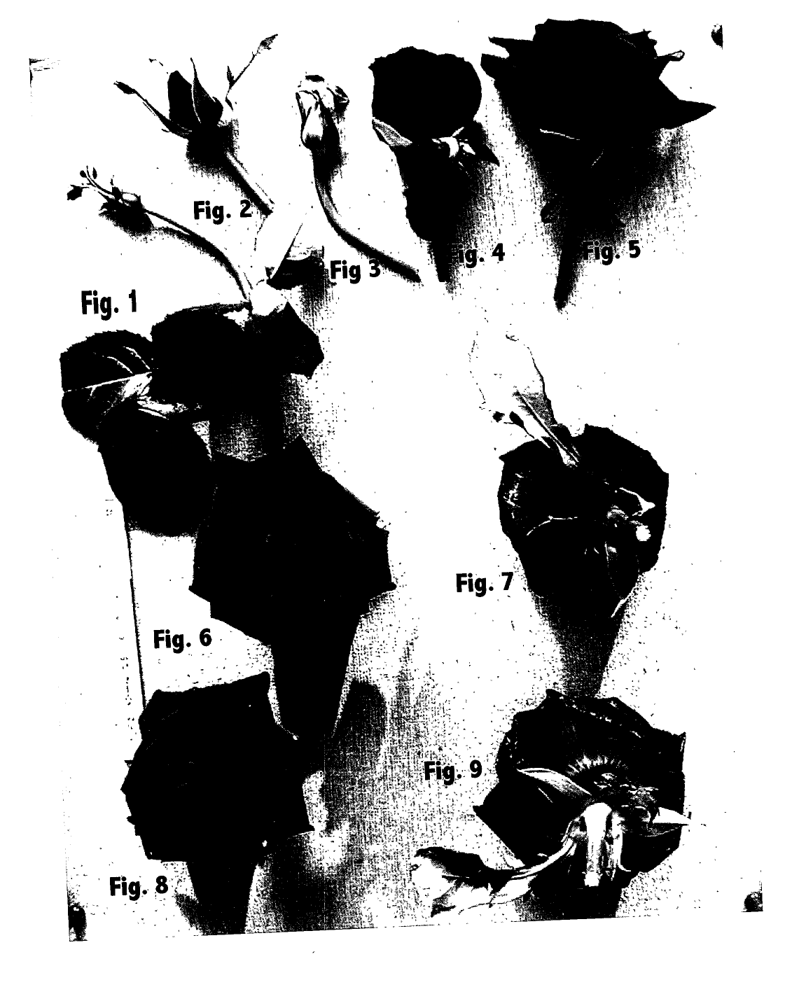

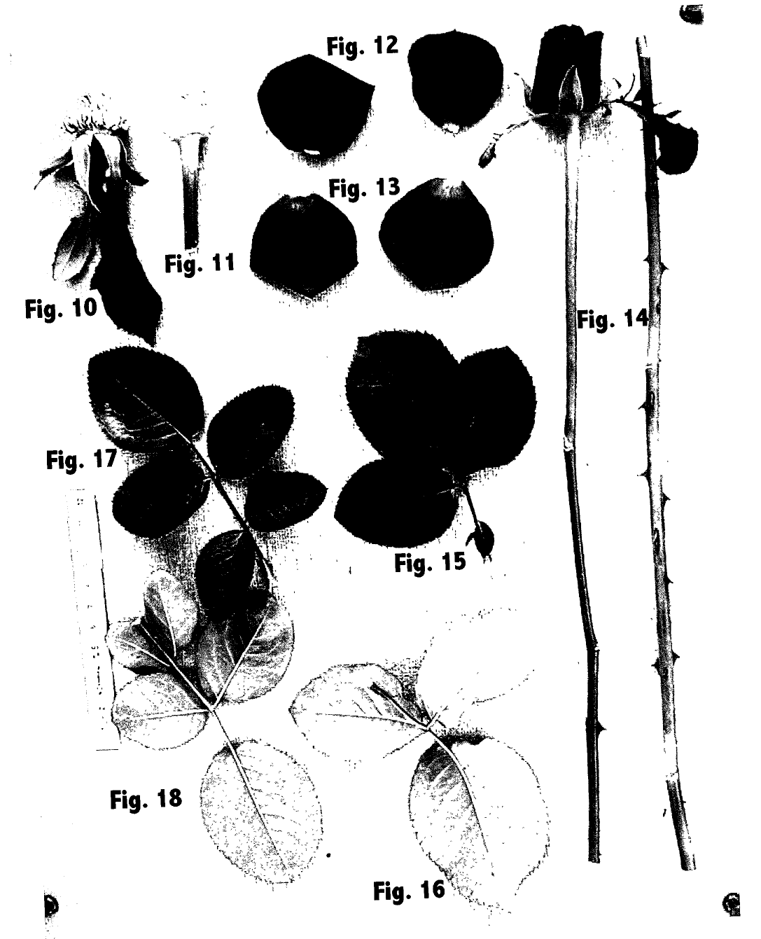

### DESCRIPTION OF THE PLANT

The following is a detailed description of ‘KORrabea’, as observed in its growth in greenhouses in Klein Offenseth-Sparrieshoop, Germany and glasshouses in California. The age of the observed plant was 11 to 13 months after propagation, produced as a cut rose plant. The cultivation at both locations was based on 18 to 26° C. temperatures. Color references are made using The Royal Horticultural Society (London, England) Colour Chart, 1995, except where common terms of color are used. For a comparison, the nearest existing rose variety is ‘JACkisca’, U.S. Plant Pat. No. 11,776.

Table 1 details several physical characteristics of ‘KORrabea’ and ‘JACkisca’.

TABLE 1

| | KORrabea | JACkisca |
|---|---|---|
| Petal color, upper surface | 46A | 53B |
| Petal color, reverse surface | 46A | 53D |
| Petal count | 40-45 | 40-45 |

### THE PLANT

Classification:

Botanical.—Rosa hybrida.

Commercial.—Hybrid tea rose.

Growth habit: Very vigorous. Grows strong upright to bushy. Production time for floral stems is generally 6 to 8 weeks depending on average temperature, light level, and cultural practices.

Height: Plants which were pruned at a height of 0.5 meter, produced floral stems 80 to 90 cm long.

Stem:

Color.—Young wood: 144A, with intonations of 178A. Older wood: 137B.

Thorns.—Incidence: Medium number of thorns. Size: 5-7 mm. Color: 178A. Shape: Deep concave, pointing downward.

Surface.—Young wood: Smooth, with prickles and stipitate glands colored 178A. Older wood: Smooth.

Stem diameter.—6-8 mm.

Internode length.—40-65 mm.

Numbers of internodes.—14-15.

### FOLIAGE

Arrangement: Alternate, compound with three to seven leaflets per leaf, generally symmetrical, abundant, and flat in aspect. Stipules at petiole base.

Quantity of leaves: 14-15 per lateral branch.

Leaf size.—Length: 110-130 mm. Width: 100-110 mm.

Petioles:

Color.—147D, with intonations of 178A.

Margins.—Entire, with stipitate glands.

Length.—3-15 mm.

Diameter.—About 1-2 mm.

Stipules:

Size.—15-20 mm.

Surface.—Smooth.

Color.—147A-147B, with intonations of 178A.

Margins.—Entire, with stipitate glands.

Rachis:

Color.—147D, with intonations of 178A.

Margins.—Margins with stipitate glands.

Length.—25-45 mm.

Leaflets:

Edge.—Serrated.

Serration.—Single.

Shape.—Ovate with acute to obtuse apex and rounded base.

Texture.—Smooth.

Appearance.—Dull.

Size.—Length: 35-85 mm. Width: 26-70 mm.

Color.—Young foliage: Upper surface: 146B, with intonations of 178A. Lower surface: 146C, with intonations of 178A. Mature foliage: Upper surface: 137A. Lower surface: 189B.

### BUD

Size upon opening:

Length.—30-35 mm.

Width.—20-25 mm.

Shape: Cupped to pointed.

Color: At one quarter opening, 45C.

### FLOWERS

Flowering characteristics: Recurrent, generally 1 bud per flowering stem.

Duration: On the plant, flowers last from 14 to 16 days. When cut and placed in a vase, flowers last from 10 to 12 days.

Fragrance: None.

Size: 55-60 mm in diameter.

Shape of flower when viewed from the side:

Upon opening.—Cupped.

Open flower.—Cupped.

Peduncle:

Color.—137A.

Texture.—Smooth, with prickles.

Length.—120-130 mm.

Angle.—Upright.

Receptacle:

Surface.—Smooth, glabrous.

Shape.—Funnel-shaped.

Size:

Height.—5-6 mm.

Width.—10-12 mm.

Color.—144A.

Sepals:

Quantity.—5.

Shape.—Narrowly ovate with acute tip.

Texture.—Leathery.

Margin.—Foliaceous appendages on 3 of the 5 sepals.

Appearance.—Dull.

Color.—Upper surface: 137C. Reverse surface: 137D.

Petals:

Color.—Upon opening: Upper surface: 46A, with intonations of 187A. Reverse surface: 46A. After opening: Upper surface: 46B. Reverse surface: 46C. General tonality of open flower: 46A on third day, 46B afterwards.

Basal petal spots.—Size: 4-6 mm. Color: 155D and 14C.

Reflex.—Outermost petals reflex backwards at opening. When fully open all petals reflex backwards.

Texture.—Smooth.

Petal edge.—Uniform.

Number per flower.—Approximately 40-45 per flower.

Petal size.—Length: 65 mm. Width: 60 mm.

Shape.—Outer petals: Round. Inner petals: Round.

Petaloids: Commonly none.

### REPRODUCTIVE ORGANS

Stamens: Approximately 150-160 on average per flower.

Pollen.—Color: 22A. Amount: Average.

Anthers.—Size: 1-2 mm. Color: 22A. Shape: Oblong.

Filaments.—Size: 8-10 mm. Color: At base: 17C. Towards anthers: 46A.

Pistils: Approximately 110-120 on average per flower.

Stigmas.—Location: Below anthers. Color: 155D.

Styles.—Color: At base is 155D, towards stigmas is 57A. Length: 2 to 3 mm.

Aptitude to bear fruit: Poor.

Resistance to diseases: Above average resistance to mildew and Botrytis under normal growing conditions in Klein Offenseth-Sparrieshoop, Germany and California.

Productivity: Commonly produces approximately 140 to 180 flowers/m²·year under greenhouse conditions.

Hips/seeds: Unknown, the plant has not been grown to the stage of seed development due to the fact that the variety is developed for use as a flowering cut rose.

Winter hardiness and drought/heat tolerance: Due to the fact that this variety is a flowering cut rose plant developed for greenhouse cultivation only, the plant has not been tested for winter hardiness or drought/heat tolerance.

I claim:

1. The new and distinct variety of hybrid tea rose plant named ‘KORrabea’ as described and illustrated.
